(19) United States
(12) Patent Application Publication
(10) Pub. No.: US 2015/0065778 A1
(43) Pub. Date: Mar. 5, 2015

(54) RADIOTHERAPY PATIENT-POSITIONING SHELL

(71) Applicant: China Xinjiang Ke Lan Shuang Yi Medical Technology Stock Co., Ltd., Urumqi (CN)

(72) Inventors: Jun YI, Urumqi (CN); Xin Jun HEI, Urumqi (CN)

(73) Assignee: China Xinjiang Ke Lan Shuang Yi Medical Technology Stock Co., Ltd., Urumqi (CN)

(21) Appl. No.: 14/016,945

(22) Filed: Sep. 3, 2013

(51) Int. Cl. A61N 5/10

(52) U.S. Cl. CPC A61N 5/10
USPC 600/1

(57) ABSTRACT

The present invention discloses a radiotherapy patient-positioning shell, which comprises a low-temperature thermoplastic board fabricated to have different positioning zones for different regions of the human body. Each positioning zone has holes. Different positioning zones respectively have different layout densities of the holes. The radiotherapy patient-positioning shell of the present invention has optimized structure and superior usability. The layout densities of different positioning zones of the radiotherapy patient-positioning shell are varied according to requirements of different regions of the human body. Thereby, different positioning zones will have different degrees of contraction and apply different intensities of fixing force to the related body regions. Thus is promoted the comfort of the user and prolonged the service life of the radiotherapy patient-positioning shell without affecting the fixing force thereof.

### BACKGROUND OF THE INVENTION

1. Field of the Invention

The present invention relates to a radiotherapy technology, particularly to a radiotherapy patient-positioning shell.

2. Description of the Related Art

The radiotherapy patient-positioning shell is normally made of a low-temperature thermoplastic board. The low-temperature thermoplastic board is placed in hot water having a temperature of 65° C. for about 1 minute to completely soften the low-temperature thermoplastic board. The completely-softened low-temperature thermoplastic board features transparency. Fully softening low-temperature thermoplastic board favors molding and fixing of the material and decreases uncomfortableness of patients during molding. The low-temperature thermoplastic board not only can memorize the shape but also can be molded repeatedly. While the low-temperature thermoplastic board, which has been stretched in molding, is not molded or fixed well, it can be softened again in 65° C. hot water. Thus, the frozen internal stress is released, and the stretched material contracts and assumes its original shape. During radiotherapy, the patient lies on a special seat, and a low-temperature thermoplastic board is used to fix the patient on the seat. The low-temperature thermoplastic board would contract for a given extent after molding, which will make the patient feel uncomfortable. In order to relieve patient's uncomfortableness, the low-temperature thermoplastic board is usually perforated to reduce the contraction thereof. At present, the commercially-available low-temperature thermoplastic board has a uniform thickness and a fixed pore diameter. The perimeter of the softened low-temperature thermoplastic board is much more easily stretched because no barrier exists in the perimeter. However, the regions where the low-temperature thermoplastic board touches the body are less likely to stretch because there are resistances existing. Thus, the low-temperature thermoplastic board experiences uneven stretch with central holes enlarged more obviously than the peripheral holes. Therefore, the fixing effect is poor, and the patient feels uncomfortable. Further, uneven hole diameters would shorten the service life of the low-temperature thermoplastic board.

### SUMMARY OF THE INVENTION

The present invention is to provide a radiotherapy patient-positioning shell, which can solve the technical problems of the conventional technology and overcome the poor fixing effect caused by uneven stretch of the low-temperature thermoplastic board.

The present invention proposes a radiotherapy patient-positioning shell, which comprises a low-temperature thermoplastic board where different positioning zones are established for different regions of the human body, wherein each positioning zone has holes, and wherein different positioning zones respectively have different layout densities of holes.

In one embodiment, the low-temperature thermoplastic board has a face positioning zone and a forehead positioning zone in addition to an auxiliary positioning zone.

In one embodiment, the low-temperature thermoplastic board has a neck positioning zone.

In one embodiment, the low-temperature thermoplastic board has shoulder positioning zones.

In one embodiment, each one of the face positioning zone and the neck positioning zone has a higher layout density of holes than the auxiliary positioning zone.

In one embodiment, the distance between two adjacent holes of each one of the face positioning zone and the neck positioning zone is smaller than that of the auxiliary positioning zone. The diameter of the holes of each one of the face positioning zone and the neck positioning zone is greater than that of the auxiliary positioning zone.

In one embodiment, the forehead positioning zone has a lower layout density of holes than the auxiliary positioning zone. The distance between two adjacent holes of the forehead positioning zone is greater than that of the auxiliary positioning zone. The diameter of the holes on the forehead positioning zone is smaller than that of the auxiliary positioning zone.

In one embodiment, the shoulder positioning zone has a lower layout density of holes than the auxiliary positioning zone.

The radiotherapy patient-positioning shell of the present invention has optimized structure and superior usability. The layout densities of different positioning zones of the radiotherapy patient-positioning shell are varied according to requirements of different regions of the human body. Thereby, different positioning zones will have different degrees of contraction and apply different intensities of fixing force to the related regions. Thus is promoted the comfort of the user and prolonged the service life of the radiotherapy patient-positioning shell without affecting the fixing force thereof.

### BRIEF DESCRIPTION OF THE DRAWINGS

FIG. 1 schematically shows the structure of a radiotherapy patient-positioning shell according to Embodiment I of the present invention;

FIG. 2 schematically shows the structure of a radiotherapy patient-positioning shell according to Embodiment II of the present invention; and

FIG. 3 schematically shows the structure of a radiotherapy patient-positioning shell according to Embodiment III of the present invention.

### DETAILED DESCRIPTION OF THE INVENTION

The radiotherapy patient-positioning shell of the present invention comprises a low-temperature thermoplastic board 1 where different positioning zones are established for different regions of the human body. Each positioning zone has holes 2. Different positioning zones respectively have different layout densities of holes 2. The layout densities of the holes 2 are varied with respect to the regions where the holes 2 exist. Thereby, different positioning zones will respectively contract to different extents and apply different fixing forces to the related body regions. Therefore, the present invention can guarantee the strength of the radiotherapy patient-positioning shell, simultaneously promoting the comfort of the person fixed by the positioning board and prolonging the service life of the radiotherapy patient-positioning shell.

### Embodiment I

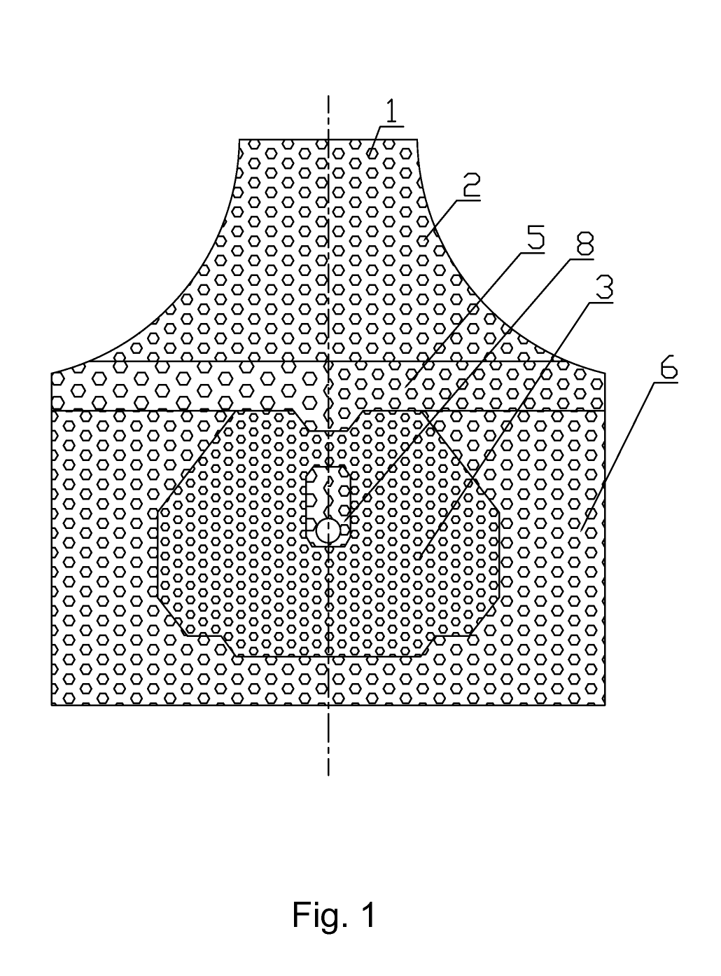

Refer to FIG. 1 for Embodiment I. Embodiment I involves a triangular head positioning shell. In Embodiment I, the low-temperature thermoplastic board 1 has a face positioning zone 3 and a forehead positioning zone 5 in addition to an auxiliary positioning zone 6.

### Embodiment II

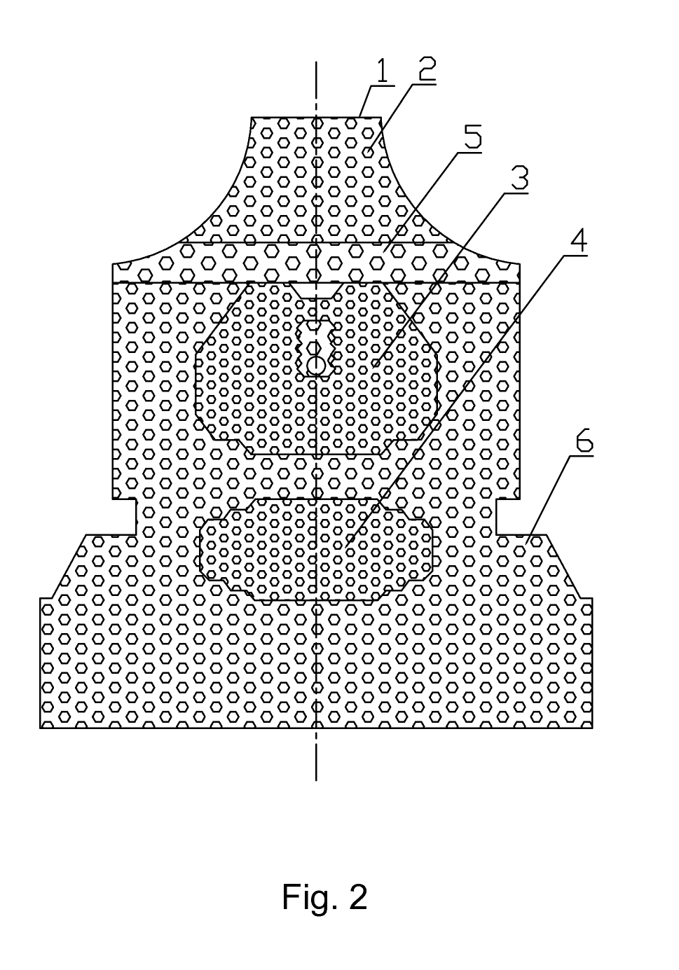

Refer to FIG. 2 for Embodiment II. Embodiment II involves an S-type perforated head positioning shell. In Embodiment II, the low-temperature thermoplastic board 1 has a neck positioning zone 4.

### Embodiment III

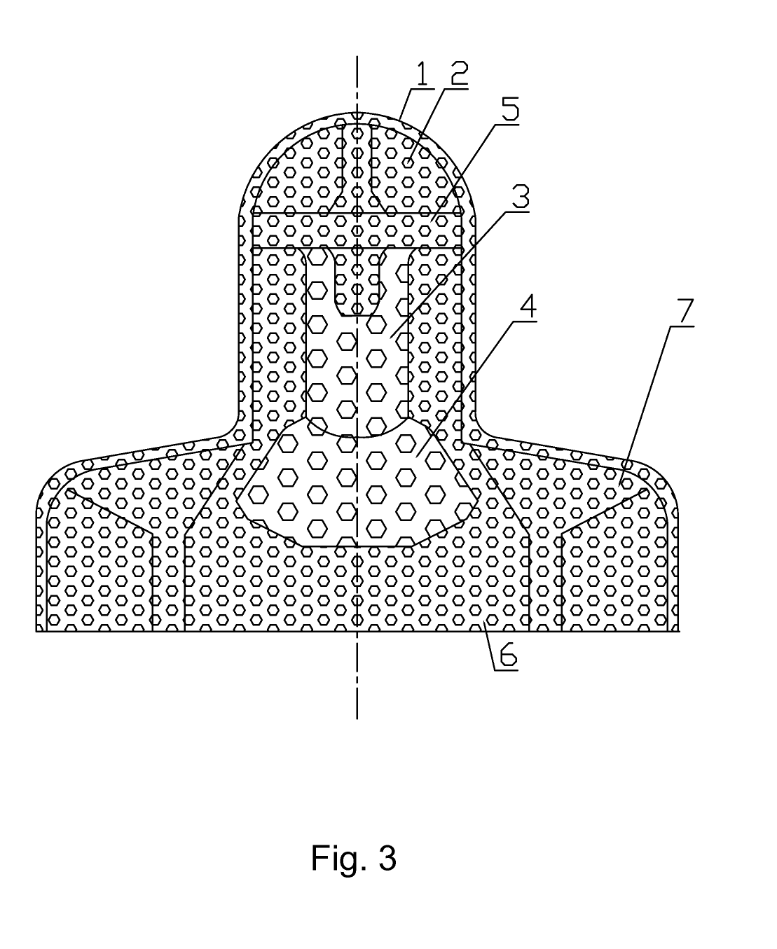

Refer to FIG. 3 for Embodiment III. Embodiment III involves an S-type perforated head-neck-shoulder positioning shell. In Embodiment III, the low-temperature thermoplastic board 1 has shoulder positioning zones 7.

In one preferred embodiment, the layout density of holes 2 of each one of the face positioning zone 3 and the neck positioning zone 4 is higher than that of the auxiliary positioning zone 6.

In one preferred embodiment, the distance between two adjacent holes 2 of each one of the face positioning zone 3 and the neck positioning zone 4 is smaller than that of the auxiliary positioning zone 6. The diameter of the holes 2 of each one of the face positioning zone 3 and the neck positioning zone 4 is greater than that of the auxiliary positioning zone 6.

In one preferred embodiment, the layout density of holes 2 of the forehead positioning zone 5 is lower than that of the auxiliary positioning zone 6. The distance between two adjacent holes 2 of the forehead positioning zone 5 is greater than that of the auxiliary positioning zone 6. The diameter of the holes 2 of the forehead positioning zone 5 is smaller than that of the auxiliary positioning zone 6.

In one preferred embodiment, the layout density of holes 2 of the shoulder positioning zone 7 is lower than that of the auxiliary positioning zone 6.

The auxiliary positioning zone 6 of the low-temperature thermoplastic board 1 can provide basic fixing strength. As the face positioning zone 3 and the neck positioning zone 4 need to cover the nose, the mouth and the Adam's apple, the face positioning zone 3 and the neck positioning zone 4 should apply less constraint force to the abovementioned regions. Patient's comfort will be promoted by increasing the layout density of the holes 2 in the face positioning zone 3 and the neck positioning zone 4, which can be realized via reducing the distance between two adjacent holes 2 and/or increasing the diameter of the holes 2. As there are bones in the forehead and the shoulders, the forehead positioning zone 5 and the shoulder positioning zone 7 may apply slightly higher pressure to the forehead and the shoulder without affecting patient's comfort obviously. Decreasing the layout density of the holes 2 in the forehead positioning zone 5 and the shoulder positioning zone 7 will apply higher pressure to the forehead and the shoulder. Decreasing the layout density of the holes 2 in the forehead positioning zone 5 and the shoulder positioning zone 7 can be realized via increasing the distance between two adjacent holes 2 and/or decreasing the diameter of the holes 2. According to requirement, ventilation holes 8, which have a diameter greater than the holes 2, may be formed on the nasal region of the face positioning zone 3.

What is claimed is:

1. A radiotherapy patient-positioning shell characterized in comprising a low-temperature thermoplastic board fabricated to have different positioning zones for different regions of a human body, wherein each one of said different positioning zones has holes, and wherein said different positioning zones respectively have different layout densities of said holes.

2. The radiotherapy patient-positioning shell according to claim 1, wherein said low-temperature thermoplastic board has a face positioning zone and a forehead positioning zone, and wherein said low-temperature thermoplastic board further has an auxiliary positioning zone in addition to said face positioning zone and said forehead positioning zone.

3. The radiotherapy patient-positioning shell according to claim 2, wherein said low-temperature thermoplastic board further has a neck positioning zone.

4. The radiotherapy patient-positioning shell according to claim 3, wherein said low-temperature thermoplastic board further has shoulder positioning zones.

5. The radiotherapy patient-positioning shell according to claim 3 or claim 4, wherein each one of said face positioning zone and said neck positioning zone has a higher layout density of said holes than said auxiliary positioning zone.

6. The radiotherapy patient-positioning shell according to claim 5, wherein a distance between two adjacent said holes of each one of said face positioning zone and said neck positioning zone is smaller than that of said auxiliary positioning zone, and wherein a diameter of said holes of each one of said face positioning zone and said neck positioning zone is greater than that of said auxiliary positioning zone.

7. The radiotherapy patient-positioning shell according to claim 2 or claim 3 or claim 4, wherein said forehead positioning zone has a lower layout density of said holes than said auxiliary positioning zone, and wherein a distance between two adjacent said holes of said forehead positioning zone is greater than that of said auxiliary positioning zone, and wherein a diameter of said holes of said forehead positioning zone is smaller than that of said auxiliary positioning zone.

8. The radiotherapy patient-positioning shell according to claim 5, wherein said forehead positioning zone has a lower layout density of said holes than said auxiliary positioning zone, and wherein a distance between two adjacent said holes of said forehead positioning zone is greater than that of said auxiliary positioning zone, and wherein a diameter of said holes of said forehead positioning zone is smaller than that of said auxiliary positioning zone.

9. The radiotherapy patient-positioning shell according to claim 6, wherein said forehead positioning zone has a lower layout density of said holes than said auxiliary positioning zone, and wherein a distance between two adjacent said holes of said forehead positioning zone is greater than that of said auxiliary positioning zone, and wherein a diameter of said holes of said forehead positioning zone is smaller than that of said auxiliary positioning zone.

10. The radiotherapy patient-positioning shell according to claim 4, wherein said shoulder positioning zones has a lower layout density of said holes than said auxiliary positioning zone.
